(19) United States
(12) Patent Application Publication
(10) Pub. No.: US 2018/0243111 A1
(43) Pub. Date: Aug. 30, 2018

(54) REINFORCED FILL-COMPOSITING PROSTHETIC APPARATUS AND METHOD OF MANUFACTURING

(71) Applicant: Extremiti3D, LLC, Johns Island, SC (US)

(72) Inventor: Barry Hand, Johns Island, SC (US)

(21) Appl. No.: 15/905,523

(22) Filed: Feb. 26, 2018

Related U.S. Application Data
(60) Provisional application No. 62/463,805, filed on Feb. 27, 2017.

(51) Int. Cl. A61F 2/80

(52) U.S. Cl. CPC A61F 2/80; B33Y 80/00

(57) ABSTRACT

An apparatus and manufacturing method for a reinforced fill-compositing prosthetic device. A prosthetic device is manufactured according to an additive manufacturing method. The prosthetic device is manufactured with a multitude of internal channels running in one or more linear or angular paths relative to a calculated sheer force direction, which may follow the contours of the prosthetic device. Channels defining an internal structure of a socket wall are open on at least one end of the prosthetic device. One or more structural inserts comprising a strength tested tensile strength material, such as carbon fiber, are inserted into the channels and bonded into place.

### CROSS-REFERENCE TO RELATED APPLICATIONS

This application claims the benefit of U.S. Provisional Patent Application Ser. No. 62/463,805, filed on Feb. 27, 2017 entitled “REINFORCED FILL-COMPOSITING PROSTHETIC DEVICE”, the disclosure of which is hereby incorporated in its entirety at least by reference.

### FIELD

The present disclosure relates to the field of 3D printed prosthetics; in particular, an apparatus and manufacturing method for a reinforced fill-compositing prosthetic device.

### BACKGROUND

There are approximately 84,500 to 114,000 new lower-limb amputations each year in the United States. Amputation rates are rising each year, in part because of the rapid increase in diabetes and also because of improvements in treating traumatic injury and vascular disease. More of the patients experiencing these problems are able to live longer but may require limb amputation in order to survive. Further, the recent wars in Iraq and Afghanistan have caused an increase in the number of servicemen and women who undergo an amputation, typically young individuals who are otherwise healthy. Because of the early age at which the amputation occurred, these individuals will be prosthesis (i.e. an externally applied device used to replace wholly, or in part, an absent or deficient limb segment) users for many years. Thus, there is a strong need to create quality prosthetic limbs for the increasing lower-limb amputee population.

The design of an effective prosthetic socket is crucial to the rehabilitation and overall health of a person with an amputated limb. This point cannot be overemphasized. Most of the time and energy a practitioner applies in making a prosthesis is spent on fabricating the socket that must be fitted to the residual limb. The prosthetic socket must be shaped so that it supports the residual limb in load tolerant areas, while avoiding irritation of sensitive regions on the limb that contact the inner surface of the socket. If these criteria are not achieved, residual limb soft tissue breakdown often occurs when the patient uses the prosthesis. The result of a poor socket fit may include painful sores, blisters, ulcers, or cysts on the residual limb that typically restrict continued prosthesis use and, in severe cases, necessitate a further amputation to a higher anatomical level which can lead to further disability. The incidence of skin breakdown in lower-limb amputees has been reported to be from 24% to 41%. Accordingly, at any one time, as many as 41% of prosthesis users may be experiencing breakdown of the tissue on the residual limb. The principal cause of such breakdown is a poorly fitting prosthetic socket.

In recent years, manufacturers of prosthetics have turned to 3D printing technologies to reduce the cost of manufacturing and provide better fitting prosthetics through the use of computer-assisted scans of a patient's residual limb in order to build better fitting sockets. While 3D printing technologies have provided a multitude of opportunities for better fitting and cheaper upper limb prosthetic devices, the materials and manufacturing process utilized in 3D printing methods fail to provide the necessary structural strength to meet the quality and strength standards required for lower limb prosthetic sockets; for example, the standards defined in ISO 10328.

Through applied effort, ingenuity, and innovation, Applicant has identified a number of deficiencies and problems with 3D printing manufacturing methods for lower limb prosthetic sockets. Applicant has developed a solution that is embodied by the present invention, which is described in detail below.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

An object of the present disclosure is a fill-composited lower limb prosthetic apparatus comprising a 3D printed socket comprising an interior surface and an exterior surface defining a socket wall, the socket wall extending from an upper perimeter, defining a proximal opening for the interior surface, to a lower perimeter defining a distal plane, the distal plane defining a circumference, the 3D printed socket being configured according to a digital scan or mold of a residual lower limb such that the interior surface of the socket wall mimics a surface of the residual lower limb, the 3D printed socket being constructed from a plurality of stratified layers; one or more apertures being disposed around at least a posterior sector of the distal plane, the one or more apertures defining a terminal end of one or more channels having side walls defining an interior structure of a lower portion of the socket wall, the interior structure of the lower portion of the socket wall being configured pursuant to the digital scan of the residual lower limb; and, one or more reinforcing strips extending through the one or more channels via the one or more apertures, the one or more reinforcing strips being bonded to the side walls of the one or more channels with an adhesive, the one or more reinforcing strips comprising carbon fiber strips or metal strips.

Another object of the present disclosure is a fill-composited lower limb prosthetic apparatus comprising a 3D printed socket comprising an interior surface and an exterior surface defining a socket wall, the socket wall extending from an upper perimeter, defining a proximal opening for the interior surface, to a lower perimeter defining a distal plane, the distal plane defining a circumference, the 3D printed socket being configured according to a digital scan of a residual lower limb such that the interior surface of the socket wall mimics a surface of the residual lower limb, the 3D printed socket being constructed from a plurality of stratified layers; one or more apertures being disposed around at least a posterior sector of the distal plane, the one or more apertures defining a terminal end of one or more channels having side walls defining an interior structure of a lower portion of the socket wall, the interior structure of the lower portion of the socket wall being configured pursuant to the digital scan of the residual lower limb; one or more reinforcing strips extending through the one or more channels via the one or more apertures, the one or more reinforcing strips being bonded to the side walls of the one or more channels with an adhesive, the one or more reinforcing strips comprising carbon fiber strips or metal strips; at least one cord channel having side walls defining an interior portion, the at least one cord channel being disposed on a posterior portion of the exterior surface and extending continuously from a first aperture disposed on a lower portion of the exterior surface, upward to an upper portion of the exterior surface, and back downward to a second aperture disposed on the lower portion of the exterior surface to define a looped pathway; and, at least one cord or cable being threaded through the looped pathway of the at least one cord channel via the first aperture and the second aperture, the at least one cord or cable being tensioned at a first end and a second end and adhesively bonded to the side walls of the cord channel.

Yet another object of the present disclosure is a fill-composited lower limb prosthetic apparatus comprising a 3D printed socket comprising an interior surface and an exterior surface defining a socket wall, the socket wall extending from an upper perimeter, defining a proximal opening for the interior surface, to a lower perimeter defining a distal plane, the distal plane defining a circumference, the 3D printed socket being configured according to a digital scan of a residual lower limb such that the interior surface of the socket wall mimics a surface of the residual lower limb, the 3D printed socket being constructed from a plurality of stratified layers; at least two apertures being disposed on at least a posterior sector of the distal plane, the at least two apertures defining a terminal end of at least two channels having side walls defining an interior structure of a lower portion of the socket wall, each of the at least two channels being configured at opposing angles relative to each other, the interior structure of the lower portion of the socket wall being configured pursuant to the digital scan of the residual lower limb; and, at least two reinforcing strips extending through the at least two channels via the one or more apertures, the at least two of reinforcing strips being bonded to the side walls of the at least two channels with an adhesive, each of the at least two reinforcing strips being configured in a substantially L-shaped configuration such that an angled portion of the at least two reinforcing strips extends toward a center point of the distal plane.

The foregoing has outlined rather broadly the more pertinent and important features of the present invention so that the detailed description of the invention that follows may be better understood and so that the present contribution to the art can be more fully appreciated. While exemplary embodiments of the present disclosure are implemented in the field of 3D printed prosthetics, the present invention may also be implemented in the case of 3D printed orthotics as well. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present invention. It should be realized by those skilled in the art that such equivalent structures do not depart from the spirit and scope of the invention as set forth in the appended claims.

### BRIEF DESCRIPTION OF DRAWINGS

The above and other objects, features and advantages of the present disclosure will be more apparent from the following detailed description taken in conjunction with the accompanying drawings, in which:

FIG. 1 is a perspective view of a reinforced fill-compositing prosthetic, according to an embodiment of the present disclosure;

FIG. 2 is a perspective view of virtual models of the prosthetic socket and the rods or channels according to an embodiment of the present disclosure;

FIG. 3 is a functional block diagram of a manufacturing method according to an embodiment of the present disclosure;

FIG. 4 is a functional block diagram of a manufacturing method according to an embodiment of the present disclosure;

FIG. 5 is a plan view of a reinforced fill-compositing prosthetic, according to an embodiment of the present disclosure;

FIG. 6 is a plan view of a reinforced fill-compositing prosthetic, according to an embodiment of the present disclosure;

FIG. 7 is a plan view of a reinforced fill-compositing prosthetic, according to an embodiment of the present disclosure;

FIG. 8 is a plan view of a reinforced fill-compositing prosthetic, according to an embodiment of the present disclosure;

FIG. 9a is a plan view of a reinforced fill-compositing prosthetic, according to an embodiment of the present disclosure; and,

FIG. 9b is an isometric projection of a reinforced fill-compositing prosthetic, according to an embodiment of the present disclosure.

### DETAILED DESCRIPTION

Exemplary embodiments are described herein to provide a detailed description of the present disclosure. Variations of these embodiments will be apparent to those of skill in the art. Moreover, certain terminology is used in the following description for convenience only and is not limiting. For example, the words “right,” “left,” “top,” “bottom,” “upper,” “lower,” “inner” and “outer” designate directions in the drawings to which reference is made. The word “a” is defined to mean “at least one.” The terminology includes the words above specifically mentioned, derivatives thereof, and words of similar import.

A prosthetic socket can be printed using a number of additive manufacturing (AM) methods. These sockets are required to be strong along all orthogonal axes. Users will apply forces and torques to the socket which may compress one side of the socket wall and extend the opposite side. AM methods vary in cost and isotropic properties. One of the more economical methods is Fused Deposition Modeling (FDM). Typically, FDM parts are strong in compression but weaker in tension. In tension, they are stronger in the plane in which the printing occurs (X-Y), but weaker in the direction perpendicular to the layers printed (Z). The reason behind this is that layers are extruded continuously along the x-y plane. There is a time delay before the next line of polymer is fused with the previously laid adjacent polymer line, when the extruder is on its way back from the end of the line. This sequence is dictated by the slicing software and with a single extruder head there is no other way to build with FDM. During the time delay before the next line is fused, the polymer hardens and cools, but must fuse with the next line of molten polymer that gets laid down. The cooler the previous line, the weaker the bond with the next line. Consequently, the delay between laying down layers along the z-axis is the longest; hence, it is the weakest axis.

In order to provide vertical (Z) direction tensile strength, embodiments of the present disclosure provide for a method and apparatus to incorporate a channel or multiple channels which run in the Z direction, or which follow the contours of the component in the general Z direction. These channels are open on at least one end. A structural insert with minimum tensile strength properties, such as carbon fiber, can be inserted into the channel and bonded into place.

The following procedure is one embodiment for the method of creating the reinforced socket:

1. Scan the target body part.

2. Convert the scan into a solid model using software known to those in the field.

3. Hollow out the virtual solid model leaving a thin wall that mimics the surface of the body part and is thickened away from the scanned surface.

4. Insert another virtual model representing the size, shape and orientation of a structural rod, to be used as a channel to insert the physical rod into after printing.

5. Remove the rod model using a Boolean Remove type operation in the software, leaving the channel.

6. Print the socket with the channels open.

7. Prior to inserting the physical rod, the adhesive used may be injected into the channel, and will bond the individual layers of the AM part to the vertical bar. The tensile strength of the bar is translated to the layers resisting tensile stress in the Z-direction trying to pull the layers apart.

8. A multitude of channels may be created radially around the shell of the socket using the method above, or incorporated into the original virtual model of the rod.

9. Alternatively the channels may be completely embedded inside the wall of the socket, providing strength needed in any radial direction.

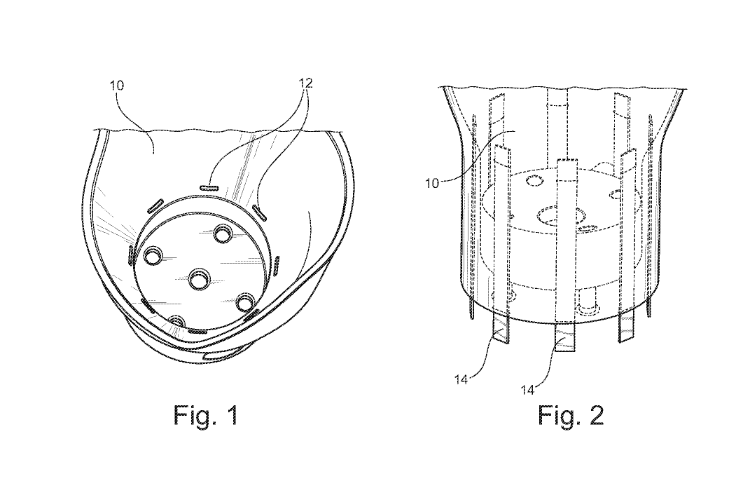

Referring now to FIGS. 1 and 2, a reinforced fill-compositing prosthetic device 10 is shown. According to an embodiment, prosthetic device 10 is a prosthetic socket. Prosthetic device 10 is manufactured according to any additive manufacturing method. Additive manufacturing, also known as 3D printing, refers to various methods used to synthesize a three-dimensional object. In 3D printing, successive layers of material are formed under computer control to create an object. In order to provide vertical (Z) direction tensile strength, prosthetic device 10 is manufactured with a multitude of channels 12 which run in the Z direction, or which follow the contours of prosthetic device 10 in the general Z direction. Channels 12 are open on at least one end of prosthetic device 10. A rod or bar 14 of strong tensile strength material, such as carbon fiber, is inserted into channels 12 and bonded into place.

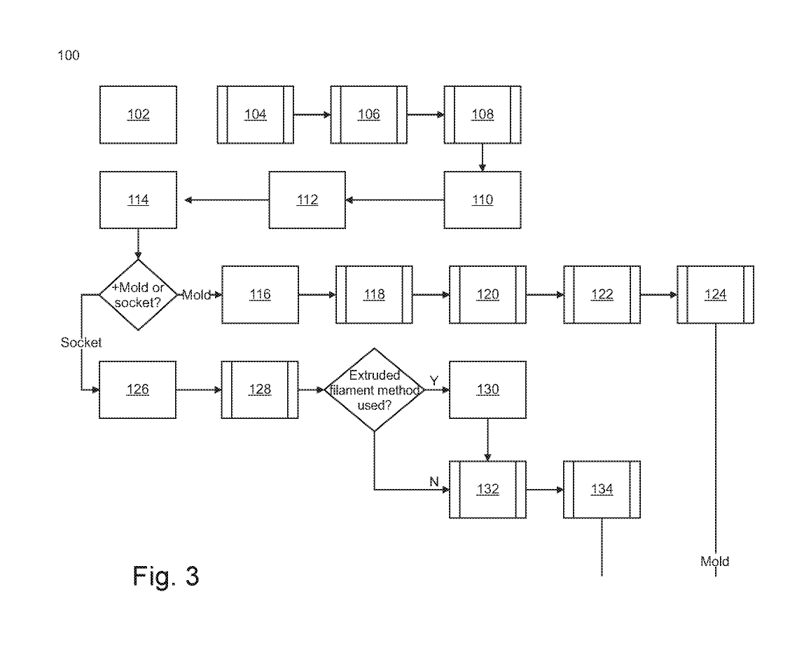

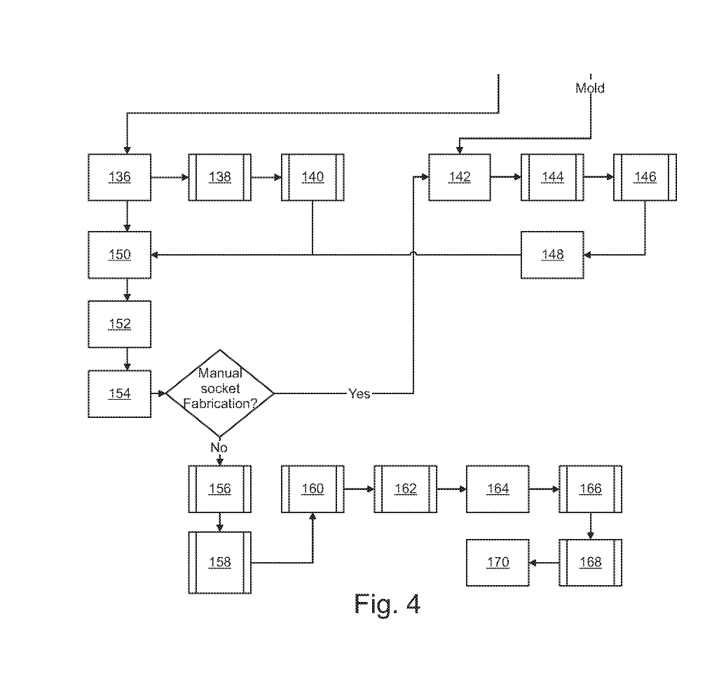

Referring now to FIGS. 3 and 4, a reinforced fill-compositing prosthetic device is manufactured according to method 100. According to an embodiment of the present disclosure, method 100 is initiated by a scan of the patient 102. In certain embodiments, the scan 102 is executed by putting on a liner and aligning a nut 104 to prepare for the scan. A patient limb is then manually scanned 106. Alternatively scan 102 could be executed by scanning a plaster mold or other type of mold that was made of the patient's limb. The resulting scan is then saved 108. The resulting scan is used to modify the 3D model in an orthotics and prosthetics (O&P) software application 110. The 3D model is then saved in an STL file format (or other relevant 3D engineering file formats) 112. The resulting STL file is then sent to a 3D printing application to prepare for additive manufacturing 114. Method 100 then selects for mold or socket. If mold is selected, then the 3D printing application converts to positive mold 116. This step is executed by adding mounting pole geometry 118, thickening the walls inward 120, optimizing the surface 122, and saving the resulting data as a printable STL file format 124. If socket is selected, then the 3D printing application converts to printed socket 126. This step is executed by thickening the socket walls outward to an appropriate thickness 128. Method 100 selects whether an extruded filament method is used. If an extruded filament method is used, then method 100 subtracts voids from socket walls to create spaces for added reinforcement 130. Method 100 then proceeds to surface optimization 132. If an extruded filament method is not used, then method 100 proceeds directly to surface optimization 132. The resulting data is then saved as a printable STL file format 134.

If proceeding with the socket, the next step in method 100 may be to 3D print a check socket 136. The socket is manufactured using any form of additive manufacturing technology used for creating models, prototypes, patterns, and production parts in a layer by layer fashion. The socket is then finished or annealed 140 to prepare for shipping to the prosthetist 150. If proceeding with the mold, the next step in method 100 is to 3D print and mold the component 142. This is executed by step 144 and finishing and step 146. The resulting socket is then thermoformed 148 and shipped to prosthetist 150. According to another exemplary embodiment, the mold is mounted on an axis and rotated. Material (plastic, carbon fiber, or other material) is deposited along the surface of the mold. For instance, nylon could be deposited first, then a layer of carbon fiber in a mesh pattern, then another layer of plastic. Carbon fiber strips may then be bonded to the first layer and printed over for the outer layer.

The 3D printed socket shipped to the prosthetist 150 is then checked 152 for fit on the patient by the prosthetist The prosthetist identifies any necessary improvements based on the fitting and modifies the file 154. If manual socket fabrication is required, then method 100 continues at the 3D print positive-mold step 142 or is manually manipulated by the prosthetist. If manual socket fabrication is not required, then method 100 uses 3D modeling software to modify socket features 156. The voids are then subtracted from the socket walls to create spaces or channels for added reinforcement 158. The outer surface may be personalized or customized 160, and the resulting data is saved as an STL file format 162. The definitive socket is 3D printed 164. Reinforcement ribs are added 166, and the socket manufacturing proceeds to finishing and annealing. The resulting socket is shipped to the prosthetist for fitting on the patient 170.

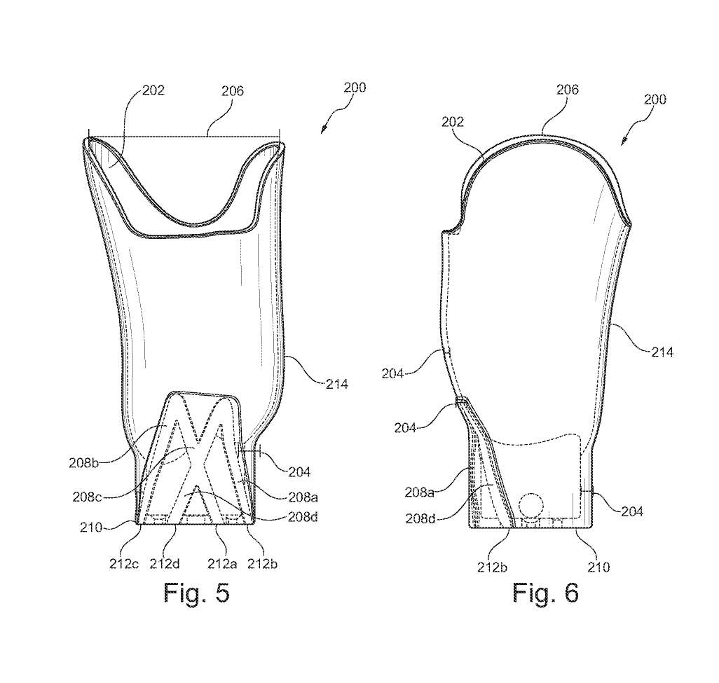

Referring now to FIG. 5, a plan view of a reinforced fill-compositing prosthetic 200 is shown. According to an embodiment, reinforced fill-compositing prosthetic apparatus 200 is a lower limb prosthetic socket for use in a prosthetic leg. A posterior plan view of reinforced fill-compositing prosthetic 200 is shown in FIG. 5. Reinforced fill-compositing prosthetic 200 is designed and manufactured according to the same design and construction techniques and materials as described in FIGS. 1-4 above. Reinforced fill-compositing prosthetic 200 is generally defined by an interior surface 202 and an exterior surface 214, and extends from an upper perimeter 206 to a distal plane 210. Distal plane 210 is circular in shape, and is configured to be coupled to the pylon or skeletal components of the prosthesis. Interior surface 202 defines an interior portion for receiving and securing a lower residual limb of a user. The contours and dimensions of interior surface 202 are defined by the scan of the user's residual limb, as discussed in FIGS. 3 and 4 above. Likewise, the contour of upper perimeter 206 is designed to provide optimal contact with an inner and outer portion of the user's thigh. A socket wall 204 is defined by the width of material between interior surface 202 and exterior surface 214. The width of socket wall 204 defines an interior structure of reinforced fill-compositing prosthetic 200. According to an embodiment, a plurality of channels 208 define the interior structure of a lower posterior portion of reinforced fill-compositing prosthetic 200. Channels 208 are defined by side walls defining a hollow interior structure extending from apertures or channel openings 212 disposed on distal plane 210 to an internal portion of socket wall 204. According to the embodiment in FIG. 5, channels 208 are comprised of congruent channels 208b and 208c and congruent channels 208a and 208d; congruent channels 208b and 208c and congruent channels 208a and 208d being configured to intersect each other to define two overlapping triangular channel paths. Channel 208b extends from aperture 212c upward at an angle to meet congruent channel path 208c, which extends from aperture 212a. Likewise, channel 208d extends from aperture 212d upward at an angle to meet congruent channel path 208a, which extends from aperture 212b. According further to the embodiment in FIG. 5, channel 208d overlaps with channel 208c to define an X-shaped internal structure within socket wall 204. In the current embodiment, apertures 212 are disposed around a posterior sector of distal plane 210 along an arc of approximately 90 degrees. Apertures 212 may be disposed adjacent to the perimeter of distal plane 210, or at another point within the width of socket wall 204, provided that channels 208 are housed in an interior portion of socket wall 204. While the configuration of channels 208 is comprised of overlapping congruent sides in FIG. 5, numerous alternative configurations for channels 208 are anticipated. For example, channels 208 may be configured vertically or at alternating and/or various angles, and may be disposed around all or part of the circumference of reinforced fill-compositing prosthetic 200. Channels 208 may be approximately 7 millimeters in width and approximately 2 millimeters in thickness. Channels 208 may be configured to extend upward at varying height; for example, channel 208 may be approximately 75 millimeters in height in a vertical configuration. It is anticipated that channels 208 may have a width in the range of about 5 millimeters to about 25 millimeters; a thickness in the range of about 2 millimeters to about 10 millimeters; and a height in the range of about 60 millimeters to about 200 millimeters. Channels 208 may also be configured to have a tapered thickness, such that the thickness of a channel 208 at an aperture 212 is greater than its thickness at an apex point of a channel 208.

Continuing with the embodiment shown in FIG. 5, a plan view of reinforced fill-compositing prosthetic apparatus 200 is shown in FIG. 6. The view of reinforced fill-compositing prosthetic apparatus 200 as shown in FIG. 6 illustrates the orientation and configuration of channels 208 relative to the total circumference of reinforced fill-compositing prosthetic apparatus 200. As shown in FIG. 6, channels 208 extend within socket wall 204 along a posterior portion of reinforced fill-compositing prosthetic apparatus 200. Channels 208 extend to a height of approximately 30% to 40% of the total height of reinforced fill-compositing prosthetic apparatus 200. It is anticipated that channels 208 may extend to a height in the range of approximately 10% to 90% of the total height of reinforced fill-compositing prosthetic apparatus 200. As shown in the current embodiment, channels 208 are disposed around a posterior sector of socket wall 204 along an arc of approximately 90 degrees; however, channels 208 may be disposed around the total circumference of socket wall 204, or any variety of dispositions therebetween. As shown in FIG. 6, channels 208 have a contoured path to match the shape of exterior surface 214.

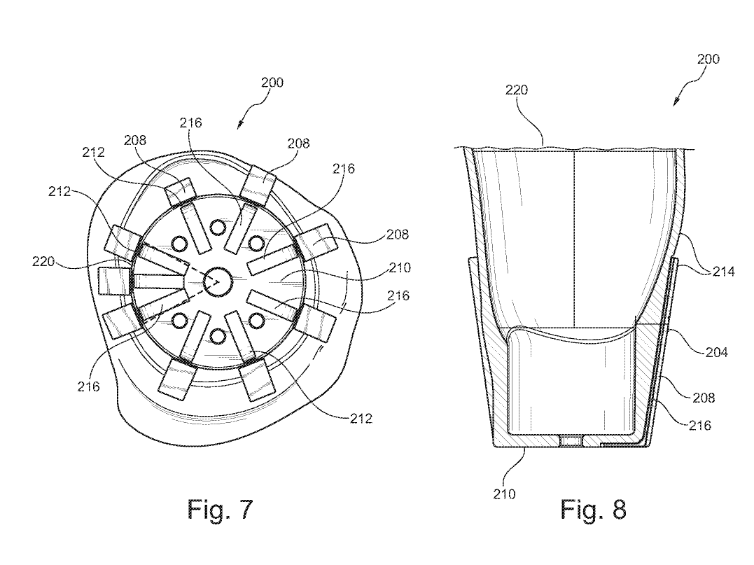

Referring now to FIG. 7, a bottom up plan view of reinforced fill-compositing prosthetic apparatus 200 is shown. According to the embodiment in FIG. 7, apertures 212 are disposed around the entire circumference of distal plane 210, and channels 208 are disposed 360 degrees around reinforced fill-compositing prosthetic apparatus 200. Apertures 212 may be spaced 45 degrees apart, relative to the circumference of distal plane 210. Apertures 212 may be configured such that nine total apertures 212 are disposed around the circumference distal plane 210, with three of the nine total apertures 212 being disposed 15 degrees apart along a 45 degree posterior sector 218 of distal plane 210. Channels 208 extend into the socket wall of reinforced fill-compositing prosthetic apparatus 200 via apertures 212 to define the internal structure of reinforced fill-compositing prosthetic apparatus 200. Numerous design alternatives are anticipated for the number and spacing of apertures 212 and channels 208, as discussed above. Structural inserts 216 are inserted into channels 208 via apertures 212. Structural inserts 216 may be configured as substantially flat strips, and may be configured as approximately 75 millimeters in height by approximately 7 millimeters in width by approximately 2 millimeters in thickness. Structural inserts 216 may be tapered in thickness; for example, structural inserts 216 may have an increasing thickness from a lower portion (the portion adjacent to aperture 212) of about 10 millimeters, to an upper portion of about 2 millimeters in thickness. Structural inserts 216 may be a variety of shapes, sizes, and angular configurations; and, in a preferred embodiment will be configured to comprise a complementary size, shape, and angular configuration to that of channels 208. In a preferred embodiment, structural inserts 216 are constructed of carbon fiber. However, numerous alternative materials that possess a similar tensile strength to weight ratio, such as certain metals, metal alloys, nanofibers, and thermoplastic composites, may be utilized for the construction of structural inserts 216. Structural inserts 216 may be configured as an L-shape, such that a substantially vertical portion of structural insert 216 may be housed in channel 208, an angular portion of structural insert 216 will interface with aperture 212, and a substantially horizontal portion of structural insert 216 will interface with the surface of distal plane 210. Structural inserts 216 are adhesively bonded to channels 208 such that structural insert 216 is “sandwiched” between the surface walls of channel 208. According to a preferred embodiment, a liquid epoxy plastic (or other commercially feasible bonding material) is injected into channels 208. Structural inserts 216 are inserted into channels 208 while the liquid epoxy plastic is still in a liquid state; after which the liquid epoxy plastic is set to cure with structural inserts 216 in place, such that structural inserts 216 are permanently bonded to the surface of channels 208. Where structural inserts 216 are configured as an L-shape, as in FIG. 7, the substantially horizontal portion of structural inserts 216 is adhesively bonded to the surface of distal plane 210. In an embodiment where structural inserts 216 are configured as an L-shape, distal plane 210 may have depressions in which the substantially horizontal portion of structural inserts 216 may interface such that the surface of distal plane 210 is flush when structural inserts 216 are bonded in place. Alternatively, an epoxy resin may be applied to the entire surface of distal plane 210 to completely encapsulate apertures 212, structural inserts 216, and the entire surface of distal plane 210. A distal plate may also be utilized to distribute the load from the prosthesis components (pylon and pyramid) to the outer walls of the socket. The distal plate may be constructed of metal, carbon fiber, or other material with similar tensile strength characteristics. In an alternative embodiment, structural inserts 216 are replaced by injecting resin with chopped fibers or other structural resin compound into channels 208. Structural resin with chopped fibers or other structural resin is allowed to harden in place and enables comparative tensile strength qualities to those of structural inserts 216.

Referring now to FIG. 8, a left side plan view of reinforced fill-compositing prosthetic apparatus 200 is shown. According to an embodiment, socket wall 204 may have an increased thickness, as compared to the embodiment shown in FIG. 6, such that a portion of structural insert 216 may extend above residual limb portion 220 without contouring to the shape of residual limb portion 220, as shown in FIG. 6. This embodiment enables structural insert 216 to provide increased tensile strength reinforcement to reinforced fill-compositing prosthetic apparatus 200, while maintaining a smooth cosmetic surface of exterior surface 214.

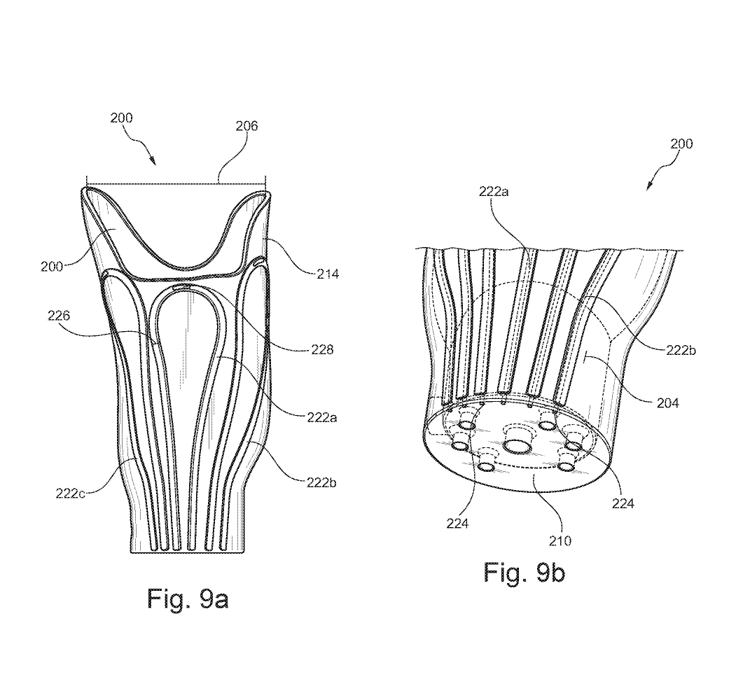

Referring now to FIG. 9a, a plan view of reinforced fill-compositing prosthetic apparatus 200 is shown. According to an embodiment, reinforced fill-compositing prosthetic apparatus 200 comprises at least one cord channel 222 running substantially the length of a posterior portion of external surface 214. According to the embodiment in FIG. 9a, reinforced fill-compositing prosthetic apparatus 200 comprises a first cord channel 222a, a second cord channel 222b, and a third cord channel 222c. Cord channels 222 are created and 3D printed internally to the walls of the socket and run vertically at acute angles to the stratified layers of reinforced fill-compositing prosthetic apparatus 200 created during 3D printing. One or more cords or cables (not shown in FIG. 9a or 9b) is threaded through each of cord channels 222a, 222b, and 222c. The one or more cords may be continuous (i.e. a single cord) or multiple. The one or more cords or cables are tensioned and bonded in place, using epoxy or a similar adhesive, thereby placing a compression load on the stratified layers of reinforced fill-compositing prosthetic apparatus 200. During use, reinforced fill-compositing prosthetic apparatus 200 is loaded such that the one or more cords or cables take the tensile forces generated by the user and help to maintain a compression throughout socket wall 204. This mitigates the tensile forces that result in delamination of the stratified layers of reinforced fill-compositing prosthetic apparatus 200.

Referring now to FIGS. 9a and 9b, according to the embodiment in FIGS. 9a and 9b, each of cord channels 222a, 222b, and 222c are configured to form a loop such that the cord ends may be secured to distal plane 210 via a cord channel aperture 224 disposed on distal plane 210. Cord channels 222a, 222b, and 222c are configured to define an internal structure within socket wall 204, and may be further defined by an external ridge 226. A cord eyelet 228 is disposed at an upper path of cord channels 222a, 222b, and 222c, and is configured to enable threading of the cord through a first cord channel aperture 224 at a first end and back through a second cord channel aperture 224 at a second end.

The present disclosure includes that contained in the appended claims as well as that of the foregoing description. Although this invention has been described in its exemplary forms with a certain degree of particularity, it is understood that the present disclosure of has been made only by way of example and numerous changes in the details of construction and combination and arrangement of parts may be employed without departing from the spirit and scope of the invention.

What is claimed is:

1. A fill-composited lower limb prosthetic apparatus comprising:
a 3D printed socket comprising an interior surface and an exterior surface defining a socket wall, the socket wall extending from an upper perimeter, defining a proximal opening for the interior surface, to a lower perimeter defining a distal plane, the distal plane defining a circumference, the 3D printed socket being configured according to a digital scan or mold of a residual lower limb such that the interior surface of the socket wall mimics a surface of the residual lower limb, the 3D printed socket being constructed from a plurality of stratified layers;
one or more apertures being disposed around at least a posterior sector of the distal plane, the one or more apertures defining a terminal end of one or more channels having side walls defining an interior structure of a lower portion of the socket wall, the interior structure of the lower portion of the socket wall being configured pursuant to the digital scan of the residual lower limb; and,
one or more reinforcing strips extending through the one or more channels via the one or more apertures, the one or more reinforcing strips being bonded to the side walls of the one or more channels with an adhesive, the one or more reinforcing strips comprising carbon fiber strips or metal strips.

2. The fill-composited lower limb prosthetic apparatus of claim 1 wherein the one or more channels are disposed equidistant around the circumference of the distal plane.

3. The fill-composited lower limb prosthetic apparatus of claim 1 wherein each reinforcing strip in the one or more reinforcing strips is tapered in thickness and/or width from a first end to a second end.

4. The fill-composited lower limb prosthetic apparatus of claim 1 further comprising a distal plate being coupled to the surface of the distal plane.

5. The fill-composited lower limb prosthetic apparatus of claim 1 wherein at least two channels in the one or more channels are configured in an overlapping configuration such that at least two strips in the one or more reinforcing strips are configured to cross each other at one or more angles.

6. The fill-composited lower limb prosthetic apparatus of claim 1 wherein the one or more reinforcing strips are configured in a substantially L-shaped configuration such that an angled portion of each reinforcing strip in the one or more reinforcing strips extends toward a center point of the distal plane.

7. The fill-composited lower limb prosthetic apparatus of claim 1 wherein the one or more reinforcing strips extend through the one or more channels at one or more opposing angles.

8. The fill-composited lower limb prosthetic apparatus of claim 5 wherein the one or more reinforcing strips are configured in a substantially L-shaped configuration such that an angled portion of each reinforcing strip in the one or more reinforcing strips extends toward a center point of the distal plane.

9. The fill-composited lower limb prosthetic apparatus of claim 6 wherein the one or more reinforcing strips are adhesively bonded to a surface of the distal plane.

10. A fill-composited lower limb prosthetic apparatus comprising:
a 3D printed socket comprising an interior surface and an exterior surface defining a socket wall, the socket wall extending from an upper perimeter, defining a proximal opening for the interior surface, to a lower perimeter defining a distal plane, the distal plane defining a circumference, the 3D printed socket being configured according to a digital scan of a residual lower limb such that the interior surface of the socket wall mimics a surface of the residual lower limb, the 3D printed socket being constructed from a plurality of stratified layers;
one or more apertures being disposed around at least a posterior sector of the distal plane, the one or more apertures defining a terminal end of one or more channels having side walls defining an interior structure of a lower portion of the socket wall, the interior structure of the lower portion of the socket wall being configured pursuant to the digital scan of the residual lower limb;
one or more reinforcing strips extending through the one or more channels via the one or more apertures, the one or more reinforcing strips being bonded to the side walls of the one or more channels with an adhesive, the one or more reinforcing strips comprising carbon fiber strips or metal strips;
at least one cord channel having side walls defining an interior portion, the at least one cord channel being disposed on a posterior portion of the exterior surface and extending continuously from a first aperture disposed on a lower portion of the exterior surface, upward to an upper portion of the exterior surface, and back downward to a second aperture disposed on the lower portion of the exterior surface to define a looped pathway; and,
at least one cord or cable being threaded through the looped pathway of the at least one cord channel via the first aperture and the second aperture, the at least one cord or cable being tensioned at a first end and a second end and adhesively bonded to the side walls of the cord channel.

11. The fill-composited lower limb prosthetic apparatus of claim 10 wherein the first aperture and the second aperture of the at least one cord channel are disposed on a posterior sector of the distal plane.

12. The fill-composited lower limb prosthetic apparatus of claim 10 wherein the one or more reinforcing strips are configured in a substantially L-shaped configuration such that an angled portion of each reinforcing strip in the one or more reinforcing strips extends toward a center point of the distal plane.

13. The fill-composited lower limb prosthetic apparatus of claim 10 wherein at least two channels in the one or more channels are configured in an X-shaped configuration such that at least two strips in the one or more reinforcing strips are configured to cross each other.

14. The fill-composited lower limb prosthetic apparatus of claim 10 wherein the one or more reinforcing strips extend through the one or more channels at one or more opposing angles.

15. The fill-composited lower limb prosthetic apparatus of claim 10 wherein the at least one cord channel runs vertically at acute angles to the plurality of stratified layers.

16. The fill-composited lower limb prosthetic apparatus of claim 10 wherein each reinforcing strip in the one or more reinforcing strips is tapered in thickness from a first end to a second end.

17. A fill-composited lower limb prosthetic apparatus comprising:
a 3D printed socket comprising an interior surface and an exterior surface defining a socket wall, the socket wall extending from an upper perimeter, defining a proximal opening for the interior surface, to a lower perimeter defining a distal plane, the distal plane defining a circumference, the 3D printed socket being configured according to a digital scan of a residual lower limb such that the interior surface of the socket wall mimics a surface of the residual lower limb, the 3D printed socket being constructed from a plurality of stratified layers;
at least two apertures being disposed on at least a posterior sector of the distal plane, the at least two apertures defining a terminal end of at least two channels having side walls defining an interior structure of a lower portion of the socket wall, each of the at least two channels being configured at opposing angles relative to each other, the interior structure of the lower portion of the socket wall being configured pursuant to the digital scan of the residual lower limb; and,
at least two reinforcing strips extending through the at least two channels via the one or more apertures, the at least two reinforcing strips being bonded to the side walls of the at least two channels with an adhesive, each of the at least two reinforcing strips being configured in a substantially L-shaped configuration such that an angled portion of the at least two reinforcing strips extends toward a center point of the distal plane.

18. The fill-composited lower limb prosthetic apparatus of claim 17 further comprising at least one cord channel having side walls defining an interior portion, the at least one cord channel being disposed on a posterior portion of the exterior surface and extending continuously from a first aperture disposed on a lower portion of the exterior surface, upward to an upper portion of the exterior surface, and back downward to a second aperture disposed on the lower portion of the exterior surface to define a looped pathway.

19. The fill-composited lower limb prosthetic apparatus of claim 18 further comprising at least one cord or cable being threaded through the looped pathway of the at least one cord channel via the first aperture and the second aperture, the at least one cord or cable being tensioned at a first end and a second end and adhesively bonded to the side walls of the cord channel.

20. The fill-composited lower limb prosthetic apparatus of claim 19 wherein the first aperture and the second aperture of the at least one cord channel are disposed on an posterior sector of the distal plane.
